(19) United States
(12) Patent Application Publication
(10) Pub. No.: US 2008/0095807 A1
(43) Pub. Date: Apr. 24, 2008

(54) Cosmetic soap

(76) Inventor: Erez Zabari, Rehovot (IL)

Correspondence Address: EDWIN TARVER, 16830 Ventura Blvd., SUITE 360, Encino, CA, 91436, US

(21) Appl. No.: 11/820,553

(22) Filed: Jun. 20, 2007

(30) Foreign Application Priority Data
Jan. 3, 2006 (IL) 172962

(51) Int. Cl. A61K 8/11; A61Q 19/10; A61K 8/97; A61K 8/98; A61K 8/92; A61K 8/72

(52) U.S. Cl. 424/401; 424/539; 424/523; 424/731; 424/739; 424/736; 424/742; 424/744; 424/747; 424/765

(57) ABSTRACT

A cosmetic soap composed of soap matrix and skin beneficial agents. The soap produced by a process comprising the steps of: storing and maintaining the previously prepared molten soap matrix in a first container at a first predetermined temperature, storing and maintaining the previously prepared beneficial agents in a second container at a second predetermined temperature; the second predetermined temperature higher than the solidification temperature of the molten soap matrix. The process further comprises the steps of: delivering the molten soap matrix and the beneficial agents to a mold wherein they are allowed to mix up, cool, and subsequently cure for a certain time, and subjecting the mixture to further conventional curing treatment thereby obtaining the final product of the cosmetic soap; the molten soap matrix and the beneficial agents are to be delivered separately from their respective containers.

### CROSS-REFERENCE TO RELATED APPLICATION

None

This U.S. national stage application claims the benefit of the priority date of Israel patent application number 172962, filed on Jan. 3, 2006, and filed as PCT application number PCT/IL2006/001449 on Dec. 17, 2006.

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The present invention relates in general to various soap bars and detergents, and more particularly to a cosmetic soap and the production process thereof. The cosmetic soap comprises encapsulated beneficial agents that help protect, moisturize, condition, and warm and/or cool the skin of a user.

However, several such soaps, detergents and their compositions and processes are known in art. For example, U.S. Pat. No. 5,188,753 to Schmidt discloses a detergent composition containing coated perfume particles encapsulated in a protective coating made of friable coating material. The encapsulant protects and preserves the perfume particles. However, in use, the friable encapsulants break so as to release and deliver the encapsulated perfume particles to the garments which are being laundered. However, the above composition is only for detergents used in laundry unlike the present invention.

In U.S. Pat. No. 5,795,852, He discloses a synthetic bar composition wherein relatively small amounts of specified hydrophobically modified polyalkylene glycol nonionic polymer are used as beneficial agents. The beneficial agents have been found to enhance mildness of bar compositions while without sacrificing processability and lather property. However, the beneficial agents are not encapsulated as in the present invention.

U.S. Pat. No. 6,248,703 to Finucane discloses extruded soap composition comprising microcapsules containing beneficial agents. The microcapsules are friable and preferably made of water insoluble materials. The microcapsules can withstand mechanical stresses and pressures typically exerted during the conventional soap extrusion processes. However, the microcapsules are capable of releasing the beneficial agents within upon washing. Although, the detergent bars use microcapsules as in the present invention, still the materials that are used in preparing the microcapsules and the beneficial agents of the present invention are different. The objects of the present invention will become better understood with reference to the appended Summary, Description, and Claims.

### SUMMARY

The present invention is a cosmetic soap comprising encapsulated skin beneficial agents embedded in a soap matrix. The beneficial agents help protect, moisturize, condition, warm or cool the skin of a user. The soap matrix and the encapsulated beneficial agents are mixed up and subjected to conventional hot or cold saponification process in a mold, then followed by further curing so as to obtain the final product of the cosmetic soap. The present invention also comprises another embodiment wherein, the beneficial agents are not encapsulated.

### DETAILED DESCRIPTION

The present invention is a cosmetic soap comprising a soap matrix and encapsulated skin beneficial agents. The soap is made of a conventional production process such as cold or hot saponification process then followed by casting. The beneficial agents help protect, moisturize, condition, warm and/or cool the skin of a user.

The soap matrix is to be made from one of the of the following elements: propylene glycol, water, sodium lauryl sulfate, sodium laureth sulfate, sodium stearate, sodium cocoate, sodium palmitate, ammonium lauryl sulfate, ammonium laureth sulfate, TEA lauryl sulfate, TEA laureth sulfate, sucrose, polyethylene glycol, glycerin, cocamidopropyl betaine and fragrances, and detergents based on isothionates, sulfosuccinate and sarcosinate, with or without pigments such as titanium dioxide. The soap matrix could also be a combination of some of the above mentioned elements.

The beneficial elements are to be made of natural and/or artificial elements that are typically used for skin care treatments such as: natural fats including jojoba, soybean, sunflower, rice bran, avocado, almond, olive, sesame, lavender, wintergreen, peach kernel oil, and castor. Essential oils such as: fish oils, menthol, jasmine, camphor, aloe Vera, white cedar, bitter orange peel, cinnamon, bergamot, citrus, calamus, pine, lavender, bay, clove, eucalyptus, lemon, thyme, peppermint, rose, sage, menthol, cineole, eugenol, citral, citronella, borneol, geraniol, evening primrose, camphor, thymol, spirantol, pinene, limonene, and terpenoil oils can also be used in the making of the beneficial elements.

The beneficial elements may also comprise waxes, such as, for example, carnauba, beeswax, lanolin, and derivatives thereof. Also, non-classified, natural ingredients, such as, crushed dried vegetables and/or fruits, honey and fruit extracts such as aloe Vera gel, powdery extracts such as papaya extract, mango extract, melon extract, vanilla planifolia extract, grape seed extract, ginseng extract, and yogurt can also be used.

Vitamins and salts such as vitamins A, B, C, D, and E, salts and minerals from the Dead Sea may also make up necessary constituents for the beneficial agent composition. Further, solvents typically used for cleansing such as polyethylene glycols, polypropylene glycols, isopropyl-palmitate, isopropyl myristate, mineral oils, silicon fluids, polysorbate and sorbitan fatty acids derivatives, and high emulsifying systems can be used as well.

Beneficial agents can also be composed of anhydrous self-warming compositions such as dehydrated zeolite, or cooling agents such as methyl lacate and any combination of essential oils typically used for cooling. Beneficial agent may also include creams consisting of water in oil emulsions typically used for skin care treatments. Beneficial agents may include a foam generating composition such as casein.

Beneficial agents can be optionally mixed with pigments for imparting ornamental effect on the soap. The pigments include ultramarines, chromium hydroxide green, iron oxides. The pigments can also be those which are typically employed in the cosmetics, food and drug industries, wherein they are colored.

The encapsulant/capsules which contain the beneficial agents are made of elements such as: gelatin, lactose, cellulose, hydroxylpropyl methylcellulose, acrylates/ammonium methacrylate copolymers, ethylene/vinyl acetate copolymer, other acrylate copolymers, polybutylene terphthalate, and ceramide. The capsules can assume various shapes such as rods, spheres, ovals, droplets, diamonds, seashells, flowers, and so on.

In order to produce the soap, the soap matrix, which is in the molten state, is taken in a first container and the encapsulated beneficial agents are taken in a second container. The molten soap matrix is maintained at a temperature of 50 to 80 degree centigrade in the first container. A predefined quantity of capsules is delivered to a mold, after which, the encapsulated beneficial agents are delivered. The molten soap matrix is again delivered to the mold after the capsules are delivered. The mold is to be completely filled up with the molten soap matrix and the encapsulated beneficial agents. The molten soap matrix and the encapsulated beneficial agents are allowed to cool and cure in the mold for sometime after which the mixture is subjected to further curing thereby finally obtaining the finished product of the cosmetic soap.

In another embodiment of present invention, the beneficial agents are not encapsulated. The production process of this embodiment of the cosmetic soap includes, storing and maintaining molten soap matrix in a first container and storing and maintaining the beneficial agents in a second container. The molten soap matrix, as in the earlier case, is maintained at a temperature of 50 to 80 degree centigrade. The beneficial agents are stored at a temperature, which is preferably higher than the solidification temperature of the soap matrix. Initially, the molten soap matrix is delivered to a mold followed by injecting/pressurizing the beneficial agents through an array of tubes, which are disposed at different levels from top and bottom of the mold. The beneficial agents and the molten soap matrix are allowed to mix-up, cool, cure, and further subjected the conventional treatment as known in the art so as to obtain the finished product of the cosmetic soap.

All features disclosed in this specification, including any accompanying claims, abstract, and drawings, may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C. § 112, paragraph 6. In particular, the use of “step of” in the claims herein is not intended to invoke the provisions of 35 U.S.C. § 112, paragraph 6.

Although preferred embodiments of the present invention have been shown and described, various modifications and substitutions may be made thereto without departing from the spirit and scope of the invention. Accordingly, it is to be understood that the present invention has been described by way of illustration and not limitation.

What is claimed is:

1. A cosmetic soap comprising:
(a) a soap matrix made of at least one element/ingredient from a group of elements comprising propylene glycol, water, sodium lauryl sulfate, sodium laureth sulfate, sodium stearate, sodium cocoate, sodium palmitate, ammonium lauryl sulfate, ammonium laureth sulfate, TEA lauryl sulfate, TEA laureth sulfate, sucrose, polyethylene glycol, glycerin, cocamidopropyl betaine, isothionates based detergents, sulfosuccinate based detergents, sarcosinate based detergents, and/or a combination thereof; and
(b) skin beneficial agents made of natural and/or artificial elements that are typically used for skin care treatments such as natural fats, essential oils, waxes, non-classified natural ingredients, vitamins, and/or a combination thereof;
the soap produced by a process comprising the steps of:
1) storing and maintaining the previously prepared molten soap matrix in a first container at a first predetermined temperature;
2) storing and maintaining the previously prepared beneficial agents in a second container at a second predetermined temperature; the second predetermined temperature higher than the solidification temperature of the molten soap matrix;
3) delivering the molten soap matrix and the beneficial agents to a mold wherein they are allowed to mix up, cool, and subsequently cure for a certain time, the molten soap matrix and the beneficial agents are delivered separately from their respective containers; and
4) subjecting the mixture to further conventional curing treatment thereby obtaining the final product of the cosmetic soap.

2. The soap of claim 1, wherein the beneficial agents can comprise at least one of the natural fats from a group consisting of: jojoba, soybean, sunflower, rice bran, avocado, almond, olive, sesame, lavender, wintergreen, peach kernel oil, and castor, and/

3. The soap of claim 1, wherein the beneficial agents can comprise at least one of the essential oils from a group consisting of: fish oils, menthol, jasmine, camphor, aloe Vera, white cedar, bitter orange peel, cinnamon, bergamot, citrus, calamus, pine, lavender, bay, clove, eucalyptus, lemon, thyme, peppermint, rose, sage, menthol, cineole, eugenol, citral, citronella, borneol, geraniol, evening primrose, camphor, thymol, spirantol, pinene, limonene, and terpenoil oils, and/or a combination thereof.

4. The soap of claim 1, wherein the beneficial agents can comprise at least one of the waxes from a group consisting of: carnauba, beeswax, lanolin, and derivatives thereof.

5. The soap of claim 1, wherein the beneficial agents can comprise at least one of the non-classified natural ingredients from a group consisting of: crushed/dried vegetables and/or fruits, honey and fruit extracts such as aloe Vera gel, powdery extracts such as papaya extract, mango extract, melon extract, vanilla planifolia extract, grape seed extract, ginseng extract, and yogurt, and/or a combination thereof.

6. The soap of claim 1, wherein the beneficial agents can comprise at least one of the vitamins from a group consisting of: vitamins A, B, C, D, E.

7. The soap of claim 1, wherein the beneficial agents can comprise salts and minerals from the Dead Sea.

8. The soap of claim 1, wherein the beneficial agents can comprise solvents typically used for cleaning.

9. The soap of claim 8, wherein at least one solvent can be selected from a group consisting of: polyethylene glycols, polypropylene glycols, isopropyl-palmitate, isopropyl myristate, mineral oils, silicon fluids, polysorbate and sorbitan fatty acids derivatives, and/or a combination thereof.

10. The soap of claim 1, wherein the beneficial agents can comprise anhydrous self-warming compositions.

11. The soap of claim 10, wherein at least one of the self-warming composition can be selected from a group consisting of dehydrated zeolite, or cooling agents such as methyl lacate.

12. The soap of claim 1, wherein the molten soap matrix is maintained at a temperature ranging 50 to 80 degree centigrade.

13. The soap of claim 1, wherein the molten soap matrix is delivered to the mold, where it is allowed to cool down for a certain time or to certain temperature, followed by delivering the beneficial agents.

14. The soap of claim 13, wherein the beneficial agents are injected or pressurized into the mold by means of an array of tubes or injectors.

15. The soap of claim 14, wherein the outlets of the injectors are disposed at different levels from top and bottom of the mold.

16. The soap of claim 1, wherein beneficial agents are encapsulated.

17. The soap of claim 16, wherein the encapsulants are friable.

18. The soap of claim 16, wherein the encapsulant is made of at least one element/ingredient from a group of elements comprising: gelatin, lactose, cellulose, hydroxylpropyl methylcellulose, acrylates/ammonium methacrylate copolymers, ethylene/vinyl acetate copolymer, other acrylate copolymers, polybutylene terphthalate, and ceramide, and/or a combination thereof.

19. The soap of claim 16, wherein a predetermined quantity of molten soap matrix is delivered to the mold first followed by the encapsulated beneficial agents, after which, the remaining quantity of molten soap matrix is delivered; the molten soap matrix and the encapsulated beneficial agents are delivered such that the mold is completely filled up with the mixture thereof.

20. The soap of claim 19, wherein the encapsulated beneficial agents are concomitantly dispersed into the mold after the predetermined quantity of molten soap matrix is delivered thereto.

21. The soap of claim 16, wherein the encapsulants break and release the beneficial agent within as the cosmetic soap is being used by a user.
